(19) United States
(12) Patent Application Publication
(10) Pub. No.: US 2018/0369460 A1
(43) Pub. Date: Dec. 27, 2018

(54) WOUND DRESSING SYSTEM

(71) Applicant: 3M INNOVATIVE PROPERTIES COMPANY, St. Paul, MN (US)

(72) Inventor: Florian Bonn, Leverkusen (DE)

(21) Appl. No.: 15/571,347

(22) PCT Filed: May 10, 2016
(86) PCT No.: PCT/IB2016/052659

§ 371 Date: Nov. 2, 2017

(30) Foreign Application Priority Data
May 11, 2015 (EP) 15167091.6

(51) Int. Cl. A61M 1/00; A61F 13/00

(52) U.S. Cl. CPC A61M 1/0088; A61M 1/0084; A61F 13/00068; A61M 1/0086; A61F 2013/00174; A61F 2013/00323; A61F 13/00063; A61M 2202/0208; A61F 2013/0017; A61F 2013/00238

(57) ABSTRACT

Device for the recovery of cell-free serum from whole blood, comprising a blood bag for the reception and the coagulation of the whole blood into a solid fraction and into a fluid containing a serum, wherein the blood bag has an outlet and a barrier in the area of the outlet which is suitable for retaining at least a major portion of the solid fraction and for the passage of said fluid, a filter module which is fluidically connected to the blood bag via the outlet, comprising an interior in which a semi-permeable membrane is arranged which subdivides the interior into a retentate space and a permeate space and allows the separation of the fluid into a serum as a permeate and into a retentate in which possible particulate components contained in the fluid remain, wherein the filter module has an inlet device for introducing the fluid into the retentate space and a permeate outlet for discharging the serum from the permeate space, and at least one receiving container for serum, comprising an inlet opening which is connected to the permeate outlet of the filter module via a connecting line.

Method for the recovery of cell-free serum from whole blood, wherein the method can be carried out by means of the described device.

The invention relates to a wound care system for introduction into a wound or for application onto a skin wound and under a wound dressing, comprising at least one first and one second capillary membrane system having a two-dimensional shape, wherein the first and the second two-dimensionally shaped capillary membrane system are connected with at least one respective supply line so that fluids, media, gases and/or other substances can be conducted through the supply line and the respective capillary membrane system.

Modern wound care pursues the goal of achieving a moist wound environment that promotes the processes active in healing. Depending on the healing phase, modern, active wound dressings therefore must be in the position to keep the wound moist to provide for an improved fluid/substance exchange, for the introduction of factors/medicines and/or for an improved fluid, secretion and/or substance removal in the wound or wounds. Applications include the use of such wound dressing systems in a soft tissue wound, an abdominal wound and on a skin wound.

A method and a device for the removal of secretion or exudate from wounds is commercially known as a V.A.C® Therapy System (KCI Co., USA). In this system, an alternating introduction of fluid into the wound and a subsequent, thus also alternating and therefore discontinuous, carrying of fluid out of the wound is provided. A foamed material introduced into the wound, which foamed material exerts forces on the wound given negative pressure, should abet the wound healing in this system.

Described in DE 10 2006 042 732 is a capillary membrane system for wound treatment in which the wound should be perfused and supplied via a hollow fiber membrane arrangement made up of up to 1000 hollow fibers, having at least one common supply line and at least one common discharge line in the sense of a through-flow of a capillary bed, and an antibiotic and growth factor perfusion should be enabled. A uniform substance distribution under continuous perfusion and under generation of a moderate negative pressure should thereby be enabled. DE 10 2006 042 732 states that additional capillary membrane systems are advantageous for an optimal supply and removal.

Although advances in wound treatment can already be achieved with the capillary membrane systems described in DE 10 2006 042 732, there is a need for simple and efficient wound care systems with which the processes necessary in wound treatment, such as flushing and disinfection, may on the one hand be performed without removal of the dressing, which processes as necessary enable a nourishment, electrolyte exchange and/or detoxification, or also a growth factor supply or a supply with antibiotics, and that allow a simple and safe handling.

In multiple instances, there is the desire to provide a suitable wound care system that on the one hand enables a sufficient cleaning of the wound and on the other hand enables the targeted application of active substances in the wound. Therefore, it is the object of the present invention to provide such a wound care system.

The object is achieved via a wound care system for treatment of a wound which comprises at least a first and a second two-dimensionally shaped capillary membrane system,
wherein the first and second two-dimensionally shaped capillary membrane systems are connected with at least one respective supply line so that fluids, media, gases and/or other substances can be conducted through the supply line and the respective capillary membrane system,
   wherein the wound care system furthermore comprises
a first dispensing container, having an outlet which is releasably connected with the first capillary membrane system via a first discharge line that comprises at least one of the supply lines of the first capillary membrane system, and via which a flushing fluid can be supplied to the first capillary membrane system,
a second dispensing container, having an outlet which is releasably connected with the second capillary membrane system via a second discharge line that comprises at least one of the supply lines of the second capillary membrane system, and via which a treatment solution can be supplied to the second capillary membrane system,
a drainage system which can be coupled with a negative pressure unit and via which fluids can be discharged from the wound to be treated.

A nearly homogeneous care of a wound is possible by means of such a wound care system, wherein the configuration of the wound care system simultaneously allows a safe and simple handling. In application, the wound care system may be placed in the wound and, for example, covered by means of a semiocclusive transparent film in order to protect the wound from drying out or against infection.

The supply lines of the capillary membrane systems are then routed out from the wound region under the film, and are connected with the first or second dispensing container outside of the wound region. The drainage system may be connected with a negative pressure unit, for example via a suitable hose that is stable against negative pressure, which hose is likewise routed out from the wound region.

The first or second two-dimensionally shaped capillary membrane system may hereby be comprised of a single capillary membrane that is arranged in a meandering shape. In this embodiment, at least one of the ends of the meandering capillary membrane is open and connected with a supply line. However, the at least one capillary membrane system may also comprise multiple capillary membranes arranged in a meandering shape, the ends of which together open into a common supply line. The first or second capillary membrane system preferably comprises a plurality of capillary membranes arranged parallel to one another.

The capillary membranes of a respective capillary membrane system, said capillary membranes being arranged parallel to one another, are embedded with their outer circumference embedded fluid-tight in the wall of a supply line, at least at one of their ends, so that a fluid connection exists between the lumen of the supply line and the lumen of the capillary membranes, and fluids, media, gases and/or other substances can be conducted through the supply line and the at least one capillary membrane system. The embedding may, for example, take place with a curable silicone material, with a polyurethane resin or with an epoxy resin. Due to their better flexibility, curable silicone materials are preferably used. In the event that the capillary membranes are embedded with only one of their ends in a supply line, the other, opposite end of the capillary membranes is closed, for example via welding or gluing. The capillary membranes may also be open at both of their ends and be embedded with both of these ends in a single supply line to one side of the arrangement, wherein the capillary membranes are then formed in a U-shape at their free end and are thereby closed there. In these instances, the capillary membranes are operated in a dead-end mode.

In particular given wider wound care systems, embodiments of the capillary membrane systems having capillary membranes arranged parallel to one another, in which the capillary membranes are open at both of their ends and are embedded in a respective supply line, are advantageous, wherein the supply lines are then preferably located at opposite sides of the [sic] the respectively two-dimensionally shaped capillary membrane system. In this instance as well, the embedding is executed so that the capillary membranes are embedded fluid-tight at their outer periphery, and a fluid connection exists between the lumen of the respective supply line and the lumen of the capillary membranes. Such an embodiment with two supply lines allows a supply and/or removal across the at least one capillary membrane system in cross-flow mode. The design of the at least one capillary membrane system with two supply lines may also be appropriate with a view towards a good homogeneity of the supply or removal across the area of the wound, in particular given broader mats or wound care systems. However, an embodiment having two supply lines also enables a simultaneous or also an alternating supply of a wound with different media via the same capillary membrane system.

The diameter of the supply lines is primarily directed toward the outer diameter of the capillary membranes embedded within them. Therefore, the at least one supply line preferably has an internal diameter in a range from 0.1 to 10 mm. It is likewise preferred if the wall thickness is in a range from 0.1 to 5 mm. In the event of the use of a supply line having non-circular cross section, the equivalent diameter d=4A/U of the internal cross section is applied with A as the area of the internal cross section, and U as its circumference. For example, the supply line may also have an oval or approximately square or rectangular internal cross section. Silicone hoses, for example, have proven to be suitable for the supply lines, through the wall of which the capillary membranes pass and in which they are cemented in place. Supply lines are preferably formed from a flexible silicone hose. The embedding or the gluing in the wall of the supply line may take place by means of typical adhesives, for example by means of curable silicone materials, polyurethane resins or an epoxy resin.

In an advantageous embodiment, the first and/or the second capillary membrane system may be designed in the form of a capillary membrane mat made up of capillary membranes arranged parallel to one another, wherein the capillary membranes in the mat are connected with one another by means of connecting elements spaced apart from and in parallel with one another, and are held at a distance from one another by the connecting elements. The connecting elements may travel transversely to the capillary membranes arranged parallel to one another, or also at a different angle. The connecting elements thereby contact the capillary membranes on their outer periphery, or entwine around these. The connecting elements have no closed flow channels along their longitudinal extent; consequently, fluids thus cannot flow through them along their longitudinal extent. The connecting elements may be adhesive strips or, for example, may also be string-like elements made up of a silicone material. In a preferred embodiment, the capillary membranes are connected to a mat by means of yarn-like connecting elements. The connecting elements are particularly preferably multifilament textile yarns. Multifilament polyester yarns, polypropylene yarns or polytetrafluorethylene yarns have particularly proven themselves as multifilament textile yarns. Best suited are hydrophilic yarns, preferably made of polyester.

In a preferred embodiment, the capillary membrane mat may be a knitted mat. In such knitted mats, the capillary membranes and the connecting threads are knitted together with one another, and the capillary membranes travel transversely to the extension direction of the capillary membrane mat. The length of the capillary membranes is determined by the mat width. In a further preferred embodiment, the capillary membrane mat is a web mat. In such web mats, the capillary membranes and the connecting threads are interwoven with one another. The capillary membranes thereby travel in the extent direction or travel direction of the capillary membrane mat, and the textile threads travel transversely to this. Knitted capillary membrane mats and capillary membrane web mats, as well as possibilities for their production, are described in DE 38 39 567, DE 43 08 850 and EP 0 442 147, for example. In particular, by means of the knitted-technology mats, mats may simply be produced in which the capillary membranes are U-shaped at their free ends and are sealed there. Such mats may be produced via meandering placement of a capillary membrane in strings parallel to one another that are connected with one another by the knit threads. After completion of the knitted mat, the U-shaped ends are separated at least on one side of said knitted mat, and the open ends of the capillary membranes that are thereby created are then embedded [in] a supply line. In the event that the U-shaped ends are separated on both sides of the knitted mat, the resulting, opposite open ends may respectively be embedded in supply lines.

In a preferred embodiment, the capillary membranes are situated within the mat in such a density that the distance of the capillary membranes from one another in the mat is 1 to 10 times the external diameter of the capillary membranes, wherein the distance is measured from the longitudinal axes of the capillary membranes. Mats are thereby preferred in which the distance of the capillary membranes from one another in the mat is 1.05 to 6 times the external diameter of the capillary membranes. Especially preferred are distances of the capillary membranes from one another in the mat in the range of 1.05 to 3 times the external diameter of the capillary membranes. In a further particularly preferred embodiment, distances of the capillary membranes from one another in the mat of more than 1.5 times the external diameter of the capillary membranes are thereby [sic]. It has been found that a certain separation of the capillary membranes from one another can be achieved with this.

At the same time, with regard to a good, homogenous care of the wound to be treated it may also be important that the capillary membranes in the capillary membrane systems are connected with one another by means of multiple connecting elements spaced relative and traveling parallel to one another to form a mat, and are held at a distance from one another by said connecting elements, and that the connecting elements are located at a defined distance from one another that preferably is within a range from 1 to 50 mm, wherein a distance in a range from 3 to 20 mm is particularly preferred and such a range from 4 to 6 mm is best suitable. Namely, it has been shown that the contact points between the capillary membranes and the connecting elements significantly promote distribution of the fluid across the area of the arrangement of the capillary membranes, for example given a care of the wound to be treated, for example with a treatment solution or a nourishment solution. Given placement of such capillary membrane systems on wounds to be treated, it was thus observed that it leads to a promotion of the exit of fluids from the capillary membranes at the contact points.

The capillary membranes of the capillary membrane systems preferably have an external diameter in a range from 200 to 1500 μm. Capillary membranes having a wall thickness in a range from 20 to 400 μm are likewise advantageous, wherein their external diameter may preferably be in the aforementioned ranges.

In the present wound care system, first and second capillary membrane system are designed for the supply or drainage of fluid media. In order to then ensure a uniform care of the wound to be treated, in a preferred embodiment the capillary membranes have a high permeability to fluids. The trans-membrane water flow of the capillary membranes is hereby preferably in a range from 0.1 to 500 cm³/(min·cm²·MPa) (from 0.01 to 50 mL/(min·cm²·bar)).

The capillary membranes of the first and/or second capillary membrane system are preferably bacteria-proof. It may hereby be ensured that no bacteria arrive in the wound via a supply of flushing fluid and/or treatment solution. Within the scope of the present invention, what is thereby understood by being bacteria-proof is that the capillary membranes have a nominal pore of 0.2 μm. The capillary membranes of the first and/or the second capillary membrane system thus preferably have a nominal pore of 0.2 μm. The nominal pore is thereby defined by the retention capability of the membrane with regard to specific microorganisms. For example, a membrane having a nominal pore of 0.2 μm retains bacteria of the species Brevundimonas diminuta, but also bacteria of the species Serratia marcescens, for which a membrane with nominal pore of 0.45 μm would be sufficient. The testing or the determination of the nominal pore sizes is described in the HIMA regulation No. 3, Vol. 4, 1982 (Health Industry Manufacturers Association), for example.

In principle, all organic polymers that are known from the prior art that are suitable to form capillary membranes are considered as materials for the capillary membranes, wherein these polymers must exhibit a good biocompatibility. Moreover, it is also required that the membrane polymer allows a sterilization of the wound care system, for example via steam sterilization, sterilization by means of γ-radiation, or sterilization by means of ethylene oxide. The organic polymers may thereby be natural polymers or polymers that were produced synthetically. Natural polymers are in particular those based on cellulosic polymers, which likewise includes polymers that have been subjected to what are known as polymer-analogous reactions. Examples of polymers based on cellulose are those made from regenerated cellulose, cellulose acetate or modified cellulose, for example cellulose ester, cellulose ether, cellulose modified with benzyl groups (benzyl cellulose) or cellulose modified with diethylaminoethyl, or mixtures of these cellulosic polymers. Furthermore, polymers based on chitin or chitosan may also be used.

Those polymers that are comprised of polyolefins, polyamides, polyacrylonitriles, polycarbonates, polyesters or sulfone polymers, as well as modifications, blends, mixtures or copolymers of these polymers that are obtained from these, may be used as synthetically produced polymers, i.e. as synthetic polymers. Preferably, those that are used are those that are based on sulfone polymers, in particular polysulfone or polyethersulfone. Additional polymers, for example polyethylene oxide, polyhydroxyether, polyethylene glycol, polyvinyl alcohol or polycaprolactone, may be admixed with the synthetic polymers as additive substances. The capillary membranes may, moreover, have a coating with an additive. Such capillary membranes preferably contain a hydrophilization agent, for example polyvinyl pyrrolidone or also hydrophilic modifications of these polymers.

The capillary membranes may be modified with a view toward specific applications, for example via coupling of functional groups, or may be coated with heparin or an antibiotic or multiple antibiotics, for example.

The form of the two-dimensionally shaped capillary membrane systems may be arbitrary in its areal extent. In the event of capillary membrane systems made up of capillary membranes parallel to one another, capillary membrane systems in their simplest embodiment have a square or rectangular shape. However, it is possible, for example in systems in which the capillary membranes are embedded in a supply line only at one of their ends, that an arc-shaped contour is formed, for example via correspondingly adapted heat cutting of the free, sealed ends of the capillary membranes parallel to one another. It is likewise possible that the arrangement made up of capillary membranes parallel to one another also has a trapezoidal contour, for example.

The present wound care system may also have additional components, for example at least one additional arrangement of capillary membranes. The capillary membranes of the additional arrangement may, for example, be membranes for oxygenation, meaning membranes via which a supply of oxygen to the wound is possible. Such membranes are disclosed in EP-A-1 144 096, EP-A-0 299 381 or DE-A-28 33 493, for example. A combination with additional two-dimensionally shaped systems or arrangements of semipermeable capillary membranes or fluid-impermeable capillaries via which a tempering or a pH value regulation may take place is also possible. The respective capillary membrane systems, and possibly additional two-dimensionally shaped systems of semipermeable capillary membranes or fluid-impermeable capillaries, may thereby be placed upon one another. However, it is also possible that, for example, the capillary membranes of different capillary membrane systems are connected with one another to form a mat, wherein the different capillary membranes are embedded with their ends in different supply lines that are preferably arranged on opposite sides of the mat. For example, such mats may be obtained via knitting together of capillary membranes with meandering placement that are arranged offset from one another, in which the U-shaped deflections of the capillary membranes are located at different positions across the mat width. By cutting the respective outlying U-shaped deflections, the capillary membranes are opened on only one respective side of the mat, and may be embedded into a supply line there.

According to the invention, in addition to the first and second capillary membrane system the wound care system comprises a drainage system by means of which a discharge from the wound is possible, for example of the flushing fluid or exudate. In one embodiment, the wound care system may have a suction sponge which possess a suction line for the flushing fluid and/or for exudate. The drainage system may also be designed as an additional capillary membrane system. However, the drainage system may preferably be at least one drainage catheter, for example in the form of a hose piece made of silicone material, for example, or of a tubule. Such a drainage catheter may have perforations in its wall via which fluids may be suctioned from the wound after connection of the drainage catheter to a negative pressure unit. The at least one drainage catheter preferably has an internal diameter in a range from 0.1 to 15 mm and a wall thickness in a range from 0.1 to 3 mm. The drainage catheter may also have a non-circular cross section. In this instance, the equivalent diameter d_{D}=4A_{D}/U_{D} of the internal cross section is applied as an internal diameter, with A_{D} as the area of the internal cross section of the drainage catheter and U_{D} as its circumference.

In one embodiment, the wound care system may furthermore comprise a pocket-like wound dressing, wherein the pocket-like wound dressing is closed at its outer edge and has a top side, an underside and a pocket interior, wherein the underside and the top side are respectively fashioned from a flat material and the underside is permeable to fluids, and wherein first and second capillary membrane system are arranged in the pocket interior. The connection of the supply lines with the respective receiving containers is hereby situated outside of the pocket-like wound dressing.

The pocket-like wound dressing with the capillary membrane systems contained therein may be inserted into a wound to be treated so that the underside is in contact with the wound. Via the capillary membrane systems, the desired fluids may be supplied to the wound, which fluids distribute in the pocket after exiting the capillary membranes and are delivered to the wound via the semipermeable pocket underside.

The pocket-like wound dressing may preferably be designed so that the connection of the capillary membranes of the capillary membrane systems with the respective supply line is located in the pocket interior, and the respective supply lines lead out from the pocket-like wound dressing via passage openings adapted fluid-tightly to their external cross section. The connection of capillary membranes with the respective supply line may likewise be arranged on the top side, outside of the pocket-like wound dressing, and the arrangement of the capillary membranes may lead out from the pocket-like wound dressing for connection with the at least one supply line via a passage opening adapted to be fluid-tight.

In the event that the wound care system comprises a pocket-like wound dressing in which are arranged first and second capillary membrane system, the capillary membrane systems extend two-dimensionally in the pocket interior. The dimensions of the capillary membrane systems respectively result from the two-dimensional extent of their external dimensions. With regard to their two-dimensional extent, first or second capillary membrane system preferably fill at least 20% and particularly preferably at least 50% of the two-dimensional extent of the pocket interior of the pocket-like wound dressing. It is particularly advantageous if, with regard to their two-dimensional extent, the first or second capillary membrane system fills at least 70% of the two-dimensional extent of the pocket interior of the pocket-like wound dressing, wherein fill degrees in a range of 90% may also be realized. It is thereby advantageous if first and second capillary membrane system are arranged in the middle of the pocket-like wound dressing.

The pocket-like wound dressing may have arbitrary contours. However, the contour is preferably round, oval, square or rectangular. Underside and top side of the pocket-like wound dressing are connected with one another at the outer edge or at the outer edges of the wound dressing, for example via welding or gluing. Among other things, silicone strips that are cured are suitable for gluing. Given rectangular or square pocket-like wound dressings, the two-dimensionally shaped capillary membrane systems arranged therein preferably likewise have a rectangular or square contour. Given round or oval pocket-like wound dressings, the capillary membrane systems located therein are advantageously likewise designed square or rectangular, wherein the aforementioned dimensions likewise apply with regard to the measurements. However, they may also be adapted to the contour of the pocket-like wound dressing, for example via correspondingly adapted welding of the non-embedded ends of the capillary membranes given capillary membrane systems having respectively only one supply line, such that an arc-shaped contour results at this edge of the capillary membrane systems.

The underside of the pocket-like wound dressing is passable, i.e. permeable, to fluids. The underside may thereby be comprised of a non-woven flat material, a lattice-like or web-like material, a perforated film or a semipermeable, microporous flat membrane. In an advantageous embodiment, the underside is comprised of a non-woven flat material or a semipermeable, microporous flat membrane. The underside preferably has a permeability to water of at least 0.1 cm³/(min·cm²·MPa) (0.01 mL/(min·cm²·bar)), and particularly preferably of at least 100 cm³/(min·cm²·MPa) (10 mL/(min·cm²·bar)) auf. An underside having a permeability to water of at least 5000 cm³/(min·cm²·MPa) (500 mL/(min·cm²·bar)) has proven to be best.

For the provided applications of the wound care system in which the wound is not only supplied with fluid via the wound care system but a removal (meaning discharge) of fluids from the wound should also take place via the drainage system, it is advantageous if openings are present in the underside, wherein the openings preferably have a diameter of at least 100 μm. Diameters of the openings of at most 10 mm are thereby preferred, and of at most 5 mm are particularly preferred. In the event that the underside is comprised of a semipermeable, microporous flat membrane, in an advantageous embodiment this has additional openings, for example in the form of perforations. In the event that the openings have a non-circular contour, the equivalent diameter D=4A/U of the opening is assumed as a diameter, with A applied as the area of the respective opening and U as its circumference. The openings may be regularly or irregularly distributed across the area of the underside, wherein a regular, homogeneous distribution is preferred. The distance between the openings may thereby be in a range from 1 to 20 mm, measured from the outer edge of the openings.

Underside and top side of the pocket-like wound dressing may be comprised of the same or different materials. However, while the underside is always permeable to fluids, the top side is preferably made from a fluid-impermeable, preferably film-like material that is connected with the underside fluid-tight at its side edges. The top side may also be a semipermeable, microporous flat membrane. In this case, however, the top side has a reduced permeability to fluids with respect to the underside, in order to ensure a distribution of supplied fluid on the underside of the pocket-like wound dressing, and therewith to the wound, upon application. In the event that underside and top side are the same or an identical semipermeable, microporous flat membrane, the underside has perforations.

In principle, the same organic polymers that were previously cited for the capillary membranes and that can be processed to form flat films or flat membranes are considered as materials for the top side or underside of the pocket-like wound dressing. Underside and/or top side of the pocket-like wound dressing are preferably made from polyolefins, polyamides, polyacrylonitrile, polycarbonates, polyester or sulfone polymers, as well as modifications, blends, mixtures or copolymers of these polymers that are obtained from these. Underside and top side particularly preferably comprise sulfone polymers as a material, wherein polysulfone or polyethersulfone are best suited.

In the preferred instance that the wound care system comprises a pocket-like wound dressing, the drainage system, preferably designed as at least one drainage catheter, leads out from the pocket-like wound dressing via a correspondingly adapted, fluid-tight passage opening, and can be connected outside of the pocket-like wound dressing with a negative pressure unit in order to thus generate a negative pressure in the pocket interior upon application. The at least one drainage catheter may be a hose piece, for example made from a silicone material, or a tubule that is arranged in the pocket interior of the wound dressing and leads out of the wound dressing via the passage opening. At the segment of the at least one drainage catheter that is located inside the pocket-like wound dressing, this drainage catheter preferably has perforations in its wall via which fluids (for example, also exudate) can be suctioned from the wound, or from the inside of the pocket-like wound dressing and from the wound, after connection of the least one drainage catheter to a negative pressure unit.

In a preferred embodiment, first and second capillary membrane system of the wound care system are identical and form a single supply capillary membrane system which is connected with at least one supply line, and the first dispenser container and the second dispenser container are connected with the supply capillary membrane system via the at least one supply line of the supply capillary membrane system. The supply capillary membrane system may thereby have two supply lines that are located at the opposite ends of its capillary membranes. In this instance, for example, the first dispenser container for flushing fluid and the second dispenser container with treatment solution may be connected with different supply lines separate from one another. However, it is also possible that the first dispenser container for flushing fluid and the second dispenser container with treatment solution are connected with only one supply line of the supply capillary membrane system, via line segments connected with one another by means of, for example, a T-joint or Y-connector. Embodiments of the supply capillary membrane system are hereby also possible in which the supply capillary membrane system is connected with only one supply line.

For application, it is preferred if the flushing fluid from the first dispenser container and the treatment solution from the second dispenser container may be supplied in alternation and separately from one another. For this, the discharge line preferably has a first control device allocated to the first dispenser container between the first dispenser container and the capillary membrane system connected with said first dispenser container. The discharge line likewise has a second control device allocated to the second dispenser container between the second dispenser container and the capillary membrane system connected with said second dispenser container, wherein first and second control device can be adjusted independently of one another. Depending on the embodiment of the wound care system, the control devices may be applied to the respective dispenser container, to the discharge lines between the dispenser containers and a T-joint or Y-connector connecting the discharge lines of said dispenser container, or to the respective supply line. In one embodiment, the control devices may be a valve by means of which the respective discharge line may be opened or closed. However, they may also be control devices by means of which the flow rate through the discharge line may be regulated to defined values.

In a preferred embodiment, first and second dispenser container are releasably connected with the respective supply line via male/female connectors. Male/female connectors Luer lock connectors [sic] are especially preferred. A hose segment may hereby be connected with the respective dispenser container, which hose section is in turn connected with the respective supply line. The connection between the respective dispenser container and the supply line, i.e. between dispenser container and supply line, between hose segment and supply line or between sub-segments of the hose segment, may also be designed as a sterile welded connection, as may be established by means of a sterile connector (for example TSCD® II Sterile Tubing Welder, Terumo Co.).

In a further advantageous embodiment, the first and/or the second dispenser container is comprised of a plurality of first sub-dispenser containers that are arranged connected in parallel with one another. The outlets of the sub-dispenser containers are respectively connected with a sub-discharge line that are [sic] connected with the supply line of the first or second capillary membrane system via a connecting element and a connecting line attached thereto. The sub-discharge lines thereby preferably, respectively have a control device by means of which the respective sub-dispenser container can be connected or disconnected, or by means of which the flow rate through the sub-discharge line may be set to defined values. Such an embodiment is, for example, advantageous in particular when, for example, autologous serum that is apportioned as aliquots to multiple sub-dispenser containers should be supplied to the wound.

As stated, in wound treatments it is often necessary that phases in which a flushing of the wound takes place alternate with phases in which an application of a treatment solution in the wound region takes place. For this, the present wound treatment system comprises a first dispenser container for a flushing fluid and a second dispenser container for a treatment solution. The treatment solutions are normally supplied to the wound region only in small quantities, whereas larger volumes of flushing solution are required for sufficient cleaning of the wound and for removal of decomposition products. Therefore, in a preferred embodiment the ratio of the volume of the first dispenser container to the volume of the second dispenser container is at least 5. The ratio is particularly preferably at least 10, and especially preferably at least 20. The absolute volumes of the first and second dispenser container depend on, among other things, the size of the wound to be treated.

Typical fluids that are suitable for wound cleaning may be considered as flushing fluid. The first dispenser container preferably contains a sodium chloride solution. Solutions with growth factors, solutions containing antibiotics or other medicines, solutions for pH value regulation, or also autologous or heterologous serum are considered to be treatment solutions. In a preferred embodiment, the second dispenser container contains a serum.

In a preferred embodiment, first and/or second dispenser containers may be chargeable with pressure. However, in application it may also be sufficient to arrange the first and/or second dispenser container at a defined vertical distance above the wound via a suitable mounting device so that the outflow from the first and/or second dispenser container and the introduction of the flushing fluid and/or of the treatment solution take place under the effect of gravity.

The following measurement methods form the basis of the characterization of the properties of the capillary membranes or flat membranes used in the wound care system:

Trans-Membrane Flow (Water Permeability) for Capillary Membranes:

A testing cell having a defined capillary membrane count and length is produced from the capillary membranes to be tested. For this, the capillary membranes are embedded at their ends on both sides in a polyurethane resin. After curing the resin, the embeddings are cut to a length of approximately 30 mm, wherein the lumens of the capillary membranes are opened by the cut. The capillary lumens in the embeddings must be tested for continuity. The free length of the capillary membranes between the embeddings is typically 120+/−10 mm. The number of capillary membranes is to be dimensioned so that a filtration area of approximately 30 cm² is provided in the testing cell under consideration of the free length and the internal diameter of the capillary membranes.

The testing cell is integrated with a testing apparatus, and tempered, ultra-filtered and demineralized water flows through them at 25° C. at a defined testing pressure (approximately 0.04 MPa (0.4 bar)). The filtered water quantity obtained during a measurement time period of 2 min, meaning the permeate generated during the measurement, is detected gravimetrically or volumetrically. The system must be flushed of air before beginning the measurement. The input and output pressure at the testing cell are measured in the testing apparatus to determine the TMF. The measurement is performed at 25° C.

The trans-membrane flow TMF is determined according to the formula (I)

$${TMF}_{W} = \frac{{V}_{W}}{Δ t \cdot {A}_{M} \cdot Δ p} [ \frac{ml}{{cm}^{2} \cdot min \cdot bar} ] \qquad ( II )$$

In which:
V_{W}=volume of water [ml] flowed through the membrane sample during the measurement time period
Δt=measurement time [min]
A_{M}=area of the membrane sample through which water flowed (typically 30 cm²)
Δp=set pressure during the measurement [MPa (bar)]

Permeability to Water of the Underside of the Pocket-Like Wound Dressing:

Disc-shaped pores are punched out of the flat material to be tested of the underside of the pocket-like wound dressing, and are clamped fluid-tight on the circumference in a suitable sample mount so that a free measurement area of 17.35 cm² results. The sample mount is located in a housing through which water charged with pressure may flow. Demineralized water, tempered to 25° C., then flows at a defined pressure between 0.01 and 0.02 MPa (between 0.1 and 0.2 bar) through the clamped sample. Water volumes that flowed through the sample during a measurement time period of 60 s are determined gravimetrically or volumetrically.

Permeability to water TMF_{W} is determined according to Formula (II)

$$TMF = \frac{{V}_{W}}{Δ t \cdot {A}_{M} \cdot Δ p} [ \frac{ml}{{cm}^{2} \cdot min \cdot bar} ] \qquad ( I )$$

In which:
V_{W}=volume of water [ml] flowed through the sample during the measurement time period
Δt=measurement time [min]
A_{M}=area of the sample through which water flowed (17.35 cm²)
Δp=set pressure during the measurement [MPa (bar)]

The invention is explained in detail using the following Figures, wherein the scope of the invention is not limited by Figures.

Shown are:

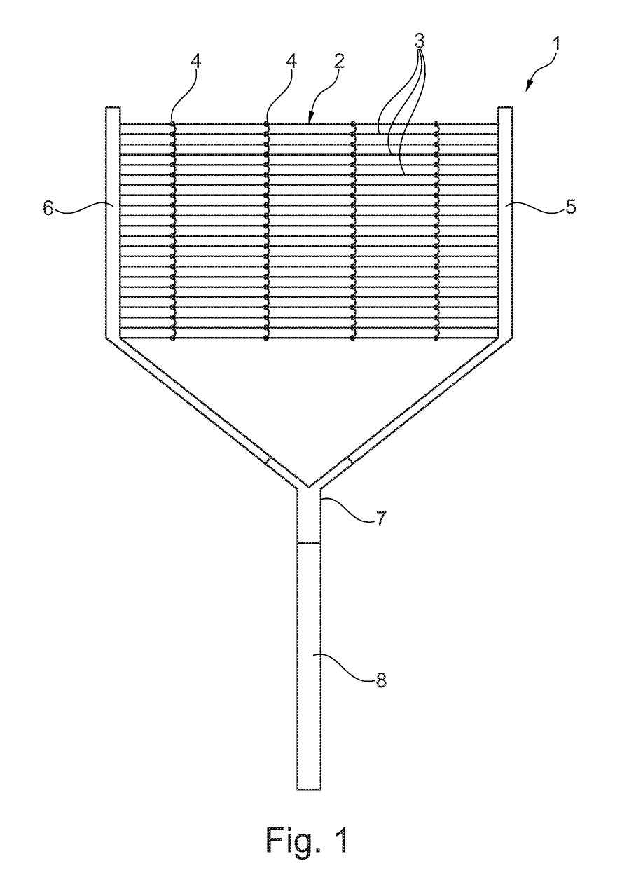

FIG. 1: capillary membrane system usable in a wound care system, having a mat made up of capillary membranes and supply lines at both ends of the mat.

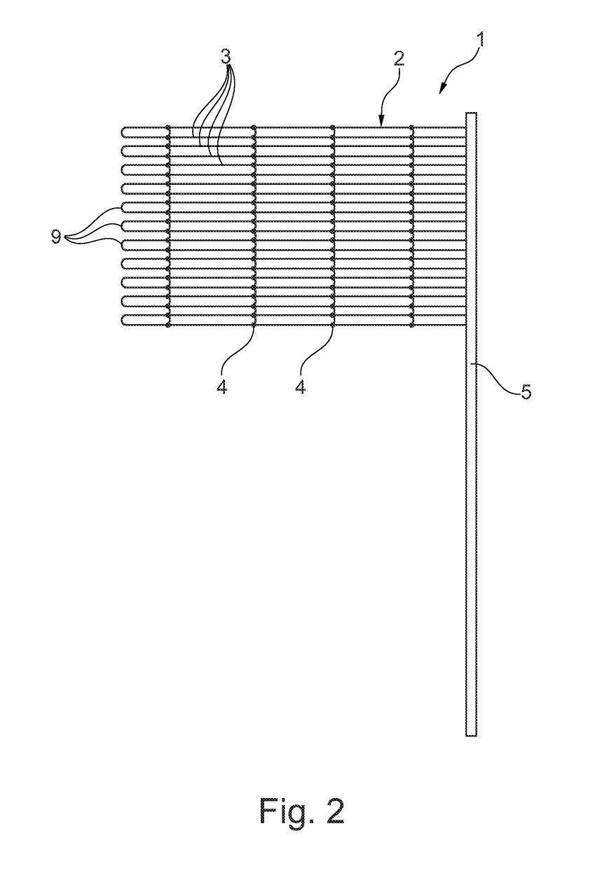

FIG. 2: capillary membrane system usable in a wound care system, with a supply line at one of the mat ends as well as U-shaped capillary membrane ends at the opposite mat end.

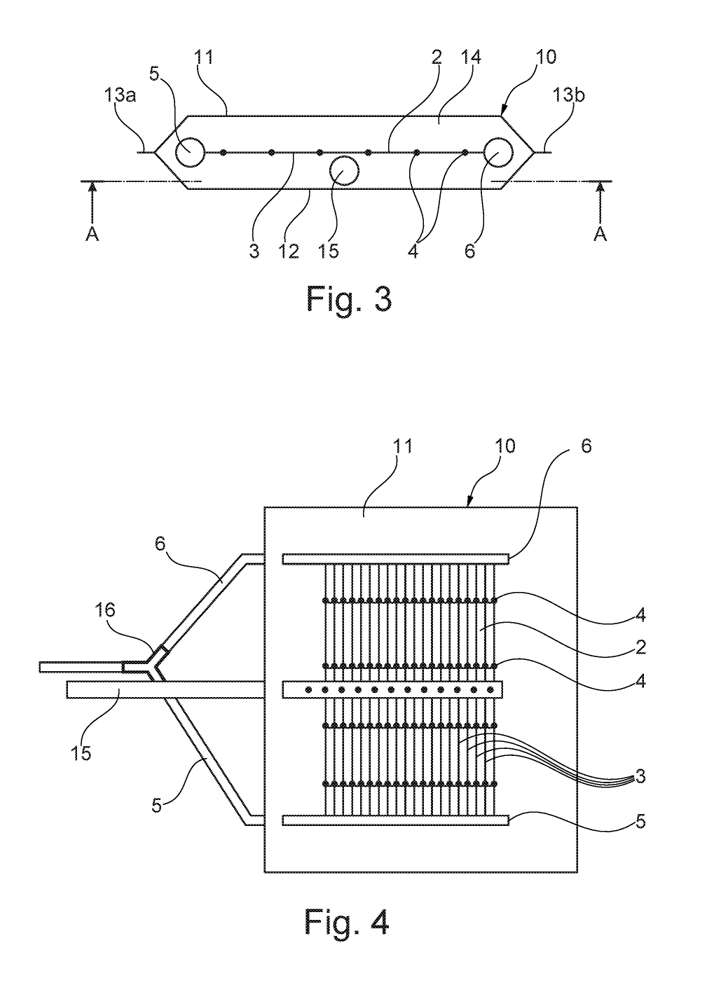

FIG. 3: cross section (schematic) through a pocket-like wound dressing system usable in a wound care system.

FIG. 4: section A-A of the pocket-like wound dressing system shown in cross section in FIG. 3.

FIG. 1 shows, schematically in plan view and not to scale, a capillary membrane system 2 made up of capillary membranes 3 that is usable in a wound care system 1 according to the invention. The capillary membranes 3 are connected by means of connecting elements 4 traveling parallel to one another to form a mat, such that they are arranged parallel to one another and held at a distance from one another. In the present example, the capillary membranes 3 are embedded with their opposite ends in supply lines 5, 6 so that a fluid connection exists between the lumens of the supply lines 5, 6 and the lumen of the capillary membranes 3. The supply lines 5, 6 are combined into a common line 8 via a Y-joint 7. From this design, it results that the flushing fluid or the treatment solution that is supplied via the line 8 is divided up among the supply lines 5, 6 and is supplied to the capillary membranes 3 in dead-end mode. Via the porous, semipermeable walls of the capillary membranes 3, the flushing fluid or the treatment solution then flows out from these and is uniformly supplied to the wound across the surface of the capillary membrane system 2.

FIG. 2 likewise shows, schematically and not to scale, a wound care system 1 in which the capillary membranes 3 are connected with only one supply line 5. The capillary membranes are open at both of their ends and embedded with both of their ends in a supply line 5. The free ends 10 of the capillary membranes 3 are U-shaped at the end of the mat that is opposite the supply line 5, and are thereby closed there. In this way, the inflow in the capillary membranes 3 of the capillary membrane system 2 shown in FIG. 3 takes place in dead-end mode.

FIG. 3 schematically shows a cross section through a pocket-like wound dressing 10 which has a top side 11 and an underside 12 that are welded with one another at their edge 13a, 13b, for example, whereby a closed pocket interior 14 is created. Presently, to simplify the presentation only a first capillary membrane system 2 is arranged in the pocket interior 14, which first capillary membrane system 2 comprises capillary membranes 3 that are connected with one another and held at a distance from one another via connecting elements 4 traveling parallel to one another, preferably in the form of multifilament yarns. In one embodiment, a required second capillary membrane system corresponding to the present invention may be arranged above or below the shown capillary membrane system 2, wherein the supply lines of the first capillary membrane system and of the second capillary membrane system may then be directed out from the pocket-like wound dressing 10 on the same side or on different sides of this.

In the present instance, the capillary membranes 3 with their opposite ends open into supply lines 5, 6 so that fluids, media, gases and/or other substances can be conducted through the supply lines 5, 6 and the capillary membrane system 2. The supply lines 5, 6 are directed out through the top side 11 of the pocket-like wound dressing 10 (not shown here).

Arranged below the two-dimensional capillary membrane system 2 is a drainage hose 15, via which exudate collecting in the wound may be removed.

FIG. 4 shows the wound dressing system presented in FIG. 3 in a cross section along the line A-A. In principle, it is a plan view from a position above the underside 12 of the pocket-like wound dressing 10 in the direction of the top side 11 of said pocket-like wound dressing 10. Arranged below the top side 11, i.e. as shown in FIG. 3, between underside 12 and top side 11, is the capillary membrane system 2 that is made up of capillary membranes 3 parallel to one another that are connected with one another and held at a distance from one another via the connecting elements 4. The capillary membranes 3 are embedded with their opposite ends in the supply lines 5, 6 so that fluids, media, gases and/or other substances can be conducted through the supply lines 5, 6 and the capillary membrane system 2. The supply lines 5, 6 are directed out of the pocket-like wound dressing 10 through the top side 11 of the pocket-like wound dressing 10, via correspondingly adapted openings in the top side 11, and in the present example are merged via a Y-connector 16 outside of the pocket-like wound dressing 10. In the present instance, the capillary membrane system 2 is thus operated in dead-end mode, meaning that a medium supplied via the supply lines 5, 6 is introduced into the capillary membrane system 2 and enters into the pocket interior entirely via the walls of the capillary membranes 3.

Also shown in FIG. 2 is the drainage hose 15 that is arranged below the capillary membrane system 2. The drainage hose has perforations in its wall so that, for example, exudate collecting in the wound may be suctioned via the drainage hose and thus removed from the wound. The drainage hose 15 is likewise directed out from the pocket-like wound dressing 10 via a correspondingly adapted opening in the top side 11 and, for example, can be connected with a negative pressure unit (not shown).

1. Device for the recovery of cell-free serum from whole blood, wherein the device comprises:
a blood bag to receive whole blood and for the coagulation of the whole blood into a solid fraction containing the cell components of the blood and into a fluid containing serum, wherein the blood bag has an outlet for the discharge of the fluid from the blood bag and a barrier in the region of the outlet, which barrier is suitable for retaining at least most of the solid fraction and for the passage of said fluid,
a filter module which is fluidically connected to the blood bag via the outlet,
wherein the filter module has a housing having an interior and an internal wall bounding the interior, in which internal wall is arranged a semipermeable membrane suitable for retaining solid fractions contained in the fluid, which membrane subdivides the interior into a retentate space and a permeate space,
wherein the semipermeable membrane enables a separation of the fluid discharged from the blood bag into serum as permeate and into a retentate in which possible particulate components contained in the fluid remain, and
wherein the filter module has an inlet device for introducing the fluid discharged from the blood bag into the retentate space, and a permeate outlet for discharging the serum from the permeate space, as well as
at least one receiving container for serum, which receiving container has an inlet opening, wherein the permeate outlet of the filter module and the inlet opening of the at least one receiving container are connected via a connecting line.

2. Device according to claim 1, characterized in that the semipermeable membrane of the filter module is a bundle of hollow fiber membranes, and the retentate space is formed by the lumens of the hollow fiber membranes, and the permeate space is formed by the external space surrounding the hollow fiber membranes and bounded by the internal wall of the housing.

3. Device according to claim 2, characterized in that the bundle is formed in a U-shape by hollow fiber membranes.

4. Device according to claim 1, characterized in that the semipermeable membrane of the filter module has a trans-membrane flow in a range from 100,000 to 400,000 dm3/(m2·h·MPa) (from 10,000 to 40,000 L/(m2·h·bar)).

5. Device according to claim 1, characterized in that the semipermeable membrane of the filter module has a nominal pore of 0.2 μm.

6. Device according to claim 1, characterized in that the filter module furthermore has a retentate outlet for discharging the retentate.

7. Device according to claim 1, characterized in that it comprises at least two receiving containers for serum that are arranged in parallel with one another, wherein the inlet openings of the receiving containers are connected via a respective connecting line with the permeate outlet of the filter module.

8. Device according to claim 1, characterized in that it comprises at least two receiving containers for serum that are arranged in series, wherein the receiving containers are fluidically connected with one another, and wherein the inlet opening of the first receiving container is connected via a connecting line with the permeate outlet of the filter module.

9. Method for the recovery of cell-free serum from whole blood, including the steps
provision of whole blood in a blood bag,
coagulation of the whole blood in the blood bag into a solid fraction containing the cell components of the blood and into a fluid containing serum,
discharging of the fluid containing the serum via an outlet of the blood bag, wherein the blood bag has a barrier in the region of the outlet, which barrier is suitable for retaining at least most of the solid fraction,
introduction of the fluid into a retentate space of a filter module via an inlet device for the introduction of the fluid,
filtering of the fluid by means of a semipermeable membrane arranged in the filter module and suitable to retain solid fractions contained in the fluid, whereby cell-free serum is obtained as a permeate and as a retentate in which solid fractions of the fluid remain,
discharging of the cell-free serum via a permeate outlet of the filter module, and
introduction of the cell-free serum into at least one receiving container via an inlet opening of the at least one receiving container, wherein the permeate outlet and the inlet opening are connected via a connecting line.

10. Method to recover cell-free serum from whole blood according to claim 9, characterized in that the outflow from the blood bag into the filter module, and furthermore from the filter module into the at least one receiving container, takes place solely under the effect of gravity.

11. Method to recover cell-free serum from whole blood according to claim 9, characterized in that a device according to claim 1 is used.
